(19) United States
(12) Patent Application Publication
(10) Pub. No.: US 2006/0076509 A1
(43) Pub. Date: Apr. 13, 2006

(54) Electron beam irradiating method and manufacturing method of magnetic recording medium

(75) Inventors: Takeshi Okino, Kanagawa-Ken (JP); Seiji Morita, Kanagawa-Ken (JP); Yoshiyuki Kamata, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOSHIBA, Tokyo (JP)

(21) Appl. No.: 11/192,048

(22) Filed: Jul. 29, 2005

(30) Foreign Application Priority Data
Sep. 30, 2004 (JP) 2004-286420

(51) Int. Cl. A61N 5/00

(52) U.S. Cl. 250/492.2

(57) ABSTRACT

The present invention is to make it possible to form a fine pattern, and improve a recording density on a magnetic recording medium and increase signal intensity. There is provided an electron beam irradiating method which irradiates an electron beam on a resist to perform irradiating using an electron beam irradiating apparatus provided with a moving mechanism which moves a state on which a substrate applied with the resist is put in one horizontal direction, and a rotating mechanism which rotates the stage. The electron beam irradiating method includes: exposing a portion once exposed while changing a deflection amount of the electron beam at least one time in the next round and rounds subsequent thereto, when exposure is performed while a deflection amount of an electron beam is being gradually changed so as to draw a concentric circle for each round.

### CROSS-REFERENCE TO RELATED APPLICATION

This application is based upon and claims the benefit of priority from prior Japanese Patent Application No. 2004-286420 filed on Sep. 30, 2004 in Japan, the entire contents of which are incorporated herein by reference.

### BACKGROUND OF THE INVENTION

1. Field of the Invention

The present invention relates to an electron beam irradiating method and a manufacturing method of a magnetic recording medium.

2. Related Art

In a technical trend to density growth in a hard disk, a medium structure of a so-called discrete type where a magnetic portion region generating magnetic signals has been defined by a non-magnetic portion has been proposed. Though a recording and reproducing system for a medium of a discrete type having data zones and servo zones has been described in JP-A-2004-110896 publication, the publication does not describe how to manufacture a medium of the discrete type.

On the other hand, U.S. Pat. No. 5,772,905 describes a technique for transferring a mold pattern with a size of 200 nm or less, so-called nano-imprint lithography, on a film. JP-A-2003-157520 publication describes a technique for transferring a pattern of a magnetic disk of a discrete type using an imprint process. In the technique disclosed in JP-A-2003-157520, a medium pattern is formed using a stamper produced from an original disk manufactured according to an electron beam lithographic technique. However, the publication does not describe a technique about the electron beam lithography.

A magnetic disk apparatus is generally provided within a casing thereof with a disk-shaped magnetic disk of a doughnut type, a head slider including a magnetic head, a head suspension assembly supporting the head slider, a voice coil motor (VCM), and a circuit board.

The magnetic disk is sectioned to concentric tracks and each track has sectors partitioned for each constant angle. The magnetic disk is attached to a spindle motor to be rotated so that various digital data elements are recorded and reproduced. Therefore, while user data tracks are disposed in a circumferential direction, servo marks for position control are disposed so as to span respective tracks. Each servo mark includes regions such as a preamble portion, an address portion, a burst portion, and the like. The servo mark may include a gap portion in addition to these regions.

It is desired that both the user data track region and the servo region are simultaneously formed on a stamper original disk used for manufacturing a magnetic disk of a discrete type utilizing to the imprint system. Otherwise, when one of the user data track region and the servo region is added to the other later, it is difficult to position the both regions, which result in necessity for a complicated manufacturing step.

In manufacture of an original disk, a pattern on the disk can be formed by exposing photosensitive resin with chemical rays such as mercury lamp rays, ultraviolet rays, electron beams, X-rays. However, since it is necessary to draw concentric circles, it is preferable that drawing is performed using electron beams which can be deflected. It is necessary to connect fine patterns such as hard disc patterns where a track pitch has a size of sub-micron meters with high dimensional accuracy. Therefore, it is desirable to employ a system where a stage is continuously moved instead of a step and repeat system where a stage is kept static during drawing operation using an electron beam, and after all patterns within one field have been drawn, the stage is moved to the next field.

It is preferable that an electron beam irradiating apparatus of a stage continuous moving system having a moving mechanism which moves a stage in one horizontal direction and a rotating mechanism which rotates the stage, as shown in FIG. 8, is selected from electron beam irradiating apparatuses which can render concentric circles and is used. In the selected electron beam irradiating apparatus, when electron beam exposure is performed by irradiating spot beams on photosensitive resin on a substrate placed on the stage from one point on a moving axis, if an electron beam is not deflected without applying an external force to the electron beam, a distance between a rotational center of the substrate and an irradiation position of an electron beam increases according to time elapsing, so that the electron beam spirals, as shown in FIG. 9. Accordingly, as shown in FIG. 10, a concentric circle can be drawn by deflecting an electron beam while gradually changing a deflection intensity (a deflection amount) for each rotation in an electron beam exposing step.

On the other hand, when such a constitution is employed that regarding a line in a radial direction, a beam is emitted or stopped only for each angular position corresponding to the line, beams are connected to draw the line. Specifically, as shown in FIG. 11, when a medium in which four servo regions s1 to s4 and four data regions d1 to d4 are disposed is produced, as shown in FIG. 12, a deflection amount is increased in the (k+1)-th round according to advancing to the servo region s1, the data region d1, the servo region s2, the data region d2, the servo region s3, the data region d3, the servo region s4, and the data region d4. In the next round, namely, the (k+2)-th round, the deflection amount is returned back to zero, and the deflection amount is increased according to advancing to the servo region s1, the data region d1, the servo region s2, the data region d2, the servo region s3, the data region d3, the servo region s4, and the data region d4. In the next round and the round subsequent thereto, namely the (k+3)-th round and the (k+4)-th round, the deflection amounts are returned back to zero, and the deflection amounts are again increased according to advancing to the servo region s1, the data region d1, the servo region s2, the data region d2, the servo region s3, the data region d3, the servo region s4, and the data region d4, where the data regions d1, d2, d3, and d4 are blanked and electron beams are irradiated on only the servo regions s1, s2, s3, and s4. When such deflection of electron beam is performed, such an exposure pattern as shown in FIG. 13 can be obtained. In fact, the servo region s1 and the data region d4 connect to each other in an annular manner, which corresponds to the pattern shown in FIG. 11.

However, in case that lines in a circumferential direction and in a radial direction are exposed at a cutting track pitch “a” in the radial direction, there is such a problem that, when the cutting track pitch “a” is small, over-exposure tends to occur in a line extending in a radial direction, as shown in FIG. 14, and when the cutting track pitch “a” is large, a line in the circumferential direction becomes thick, if the line is obtained through a plurality of exposures.

When a line in the circumferential direction is formed through only one exposure, namely, one round exposure in order to make the line thin, there occurs such a problem that lack in exposure amount occurs in a line extending in a circumferential direction or excess in exposure amount occurs in a line extending in a radial direction, which results in impossibility in formation of a fine pattern.

In a pattern with a poor rectangular shape, there may occur such a problem that imprinting can not be conducted with an excellent contrast or a high pressure is required at a time of imprinting. When a medium is manufactured using the imprinting process, it is desired that, when an exposure portion formed by an electron beam constitutes a non-magnetic portion, a groove of a discrete track formed from non-magnetic material is formed to be thin and have an excellent rectangular shape in view of increase in recording density or signal intensity.

### SUMMARY OF THE INVETION

The present invention has been made in view of these circumstances, and an object thereof is to provide an electron beam irradiating method which allows formation of a fine pattern and a manufacturing method of a magnetic recording medium using the electron beam irradiating method.

According to a first aspect of the present invention, there is provided an electron beam irradiating method which irradiates an electron beam on a resist to perform irradiating using an electron beam irradiating apparatus provided with a moving mechanism which moves a state on which a substrate applied with the resist is put in one horizontal direction, and a rotating mechanism which rotates the stage, the method including: exposing a portion once exposed while changing a deflection amount of the electron beam at least one time in the next round and rounds subsequent thereto, when exposure is performed while a deflection amount of an electron beam is being gradually changed so as to draw a concentric circle for each round.

The deflection amount of an electron beam when exposure is performed while changing a deflection amount of the electron beam at least one time in the next round and rounds subsequent thereto is changed such that an exposed image before the portion once exposed is again exposed can substantially overlap with an exposed image obtained by performing exposure while changing the deflection amount of electron beam at least one time in the next round and one round subsequent thereto.

The deflection amount of an electron beam when exposure is performed while changing the deflection amount of the electron beam at least one time in the next round and rounds subsequent thereto is changed such that an exposed image before the portion once exposed is again exposed can partially overlap with an exposed image obtained by performing exposure while changing the deflection amount of electron beam at least one time in the next round and rounds subsequent thereto.

When exposed images are caused to overlap with each other, it is desirable for pattern formation to adjust a deflection amount of an electron beam such that an exposure amount of a pattern becomes symmetrical regarding a section in a radial direction.

According to a second aspect of the present invention, there is provided a manufacturing method of magnetic recording medium that manufactures a magnetic recording medium having at least a servo region and a data region, where adjacent tracks for the data region are separated from each other by a non-magnetic region portion, the manufacturing method being implemented according to an imprint process and including: conducting a portion of a resist original disk for manufacturing a stamper used for the imprint process which corresponds to the data region portion by utilizing the electron beam irradiating method above-described.

### BRIEF DISCRIPTION OF THE DRAWINGS

FIG. 1 is a diagram showing a relationship between the number of cutting rounds and a deflection amount in an electron beam irradiating method according to an embodiment of the present invention;

FIG. 2 is a diagram showing an exposure example when an electron beam irradiating is performed according to a deflection amount shown in FIG. 1;

FIGS. 3A to 3F are sectional views of manufacturing steps performed when a magnetic recording medium of a discrete type is manufactured using the electron beam irradiating method according to the embodiment of the invention;

FIGS. 4A to 4F are sectional views of manufacturing steps performed when the magnetic recording medium of a discrete type is manufactured using the electron beam irradiating method according to the embodiment of the invention;

FIG. 5 is an upper view of a specific example of a magnetic recording medium of a discrete type;

FIG. 6 is a diagram showing a relationship between the number of cutting rounds and a deflection amount in the electron beam irradiating method according to a modification of the embodiment of the invention;

FIG. 7 is a diagram showing an example of exposure performed when electron beam irradiating is performed according to the deflection amount shown in FIG. 6;

FIG. 8 is a diagram showing a relationship between movement of a stage and an electron beam in an electron beam irradiating apparatus of a stage continuous moving system;

FIG. 9 is a diagram showing an example of exposure performed without deflecting an electron beam;

FIG. 10 is a diagram showing an example of an exposure performed when an electron beam has been deflected in order to draw a concentric circle;

FIG. 11 is an upper view of a specific example of a magnetic recording medium of a discrete type;

FIG. 12 is a diagram showing a relationship between the number of cutting rounds and a deflection amount in a conventional electron beam irradiating method;

FIG. 13 is a diagram of an example of exposure performed when electron beam irradiating is conducted according to the deflection amount shown in FIG. 12;

FIG. 14 is a diagram of an example of exposure performed in a state that a cutting track pitch is small;

FIG. 15 is a diagram of an example of exposure performed in a state that a cutting track pitch is large; and

FIGS. 16A to 16D are sectional views of manufacturing steps showing a manufacturing method of a magnetic disk medium of a discrete type according to a third example of the present invention.

### DETAILED DESCRIPTION OF THE INVENTION

### Embodiments

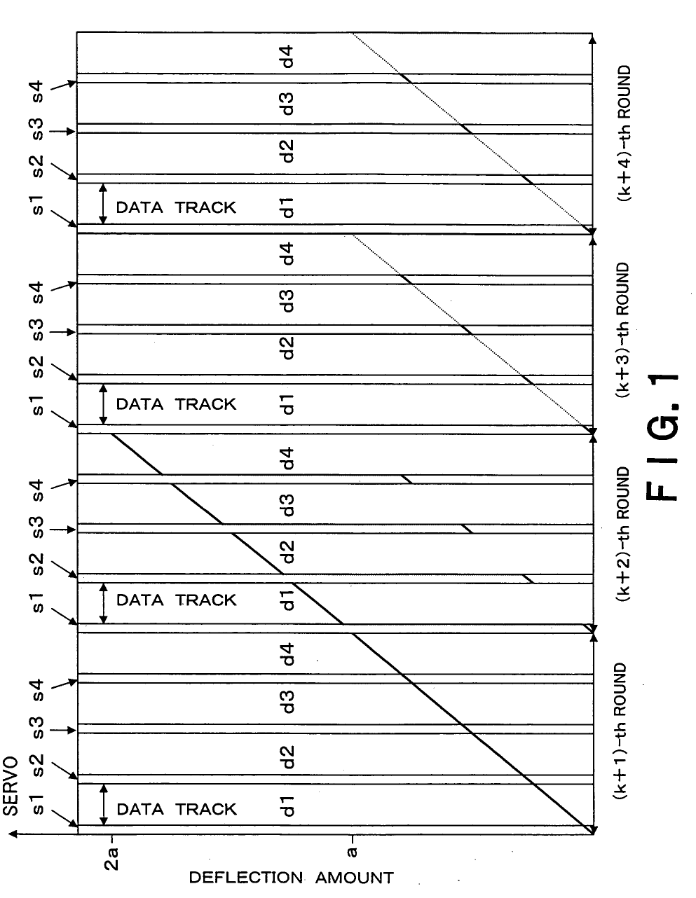

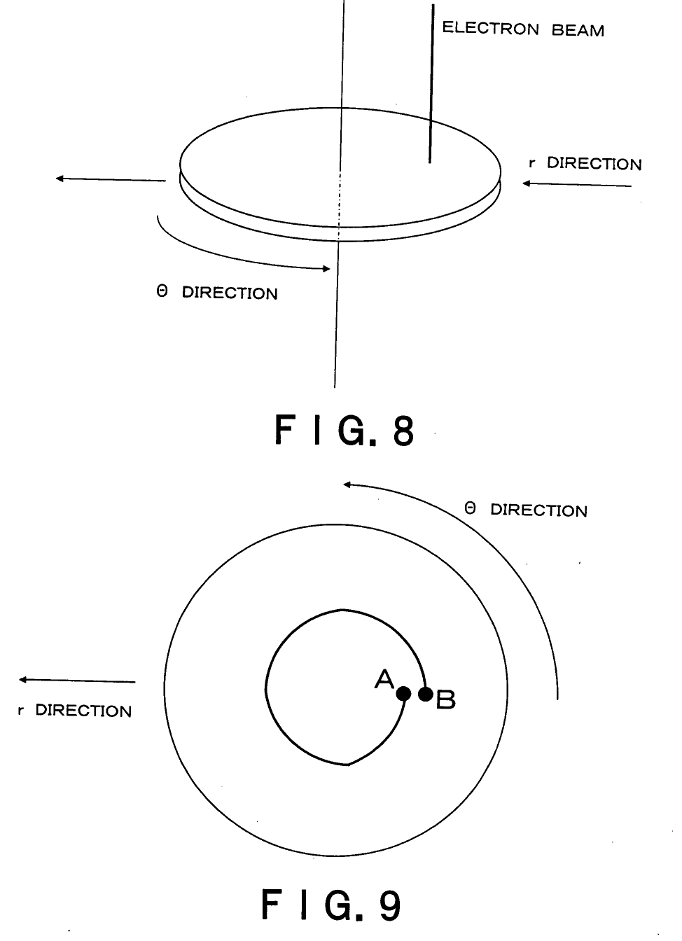

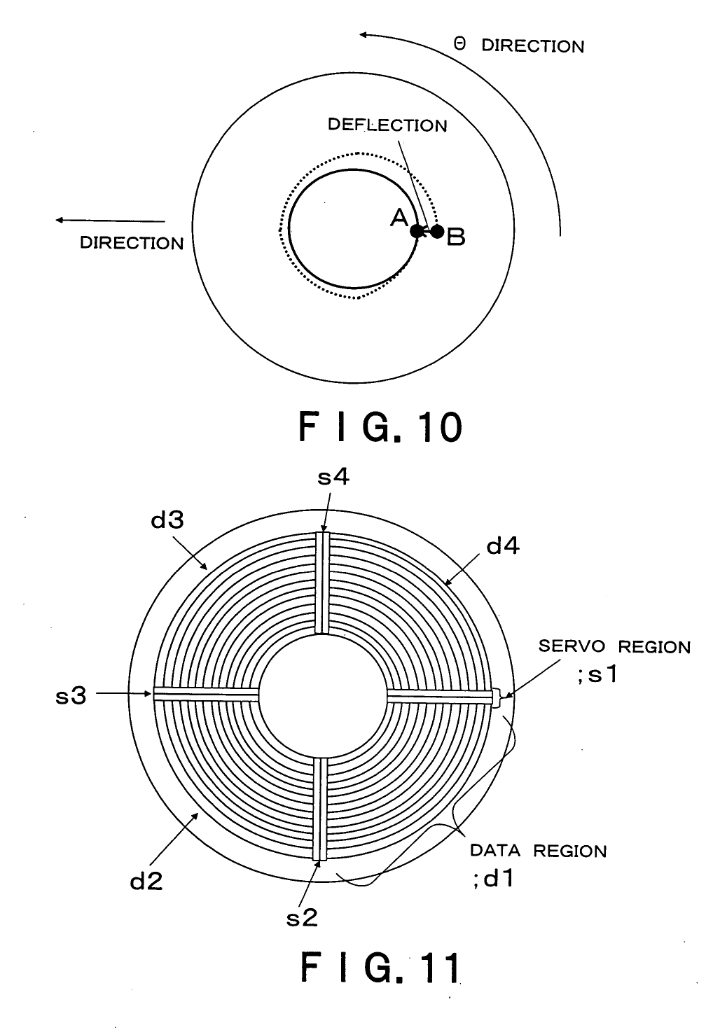

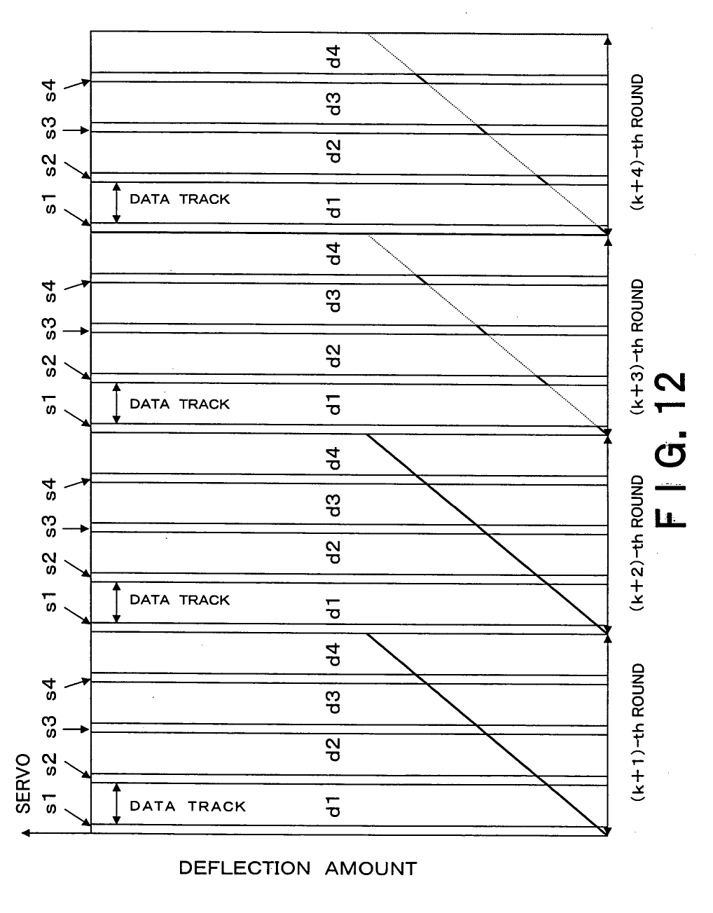

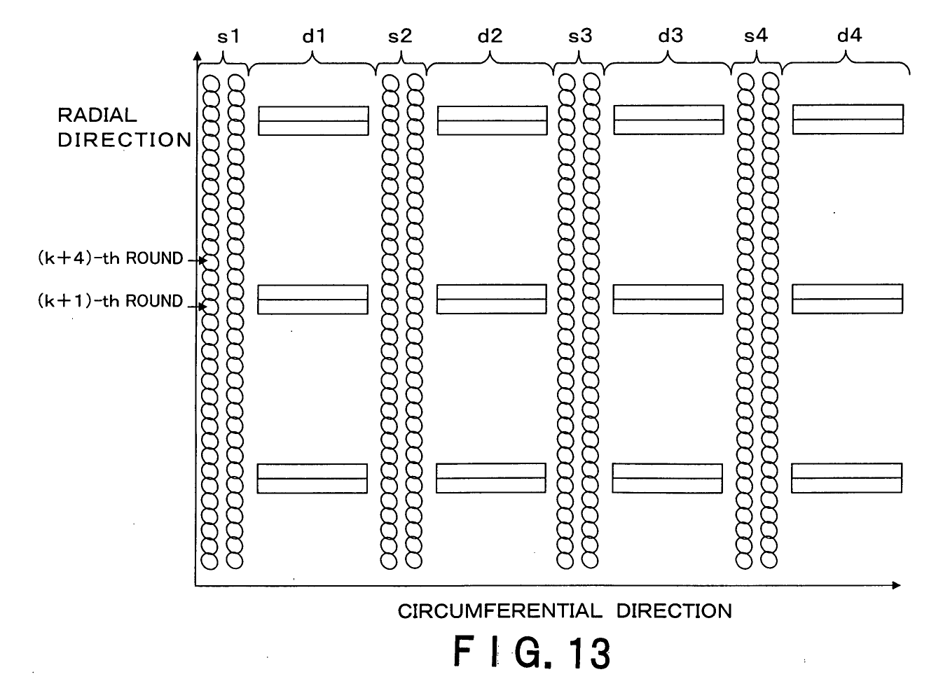

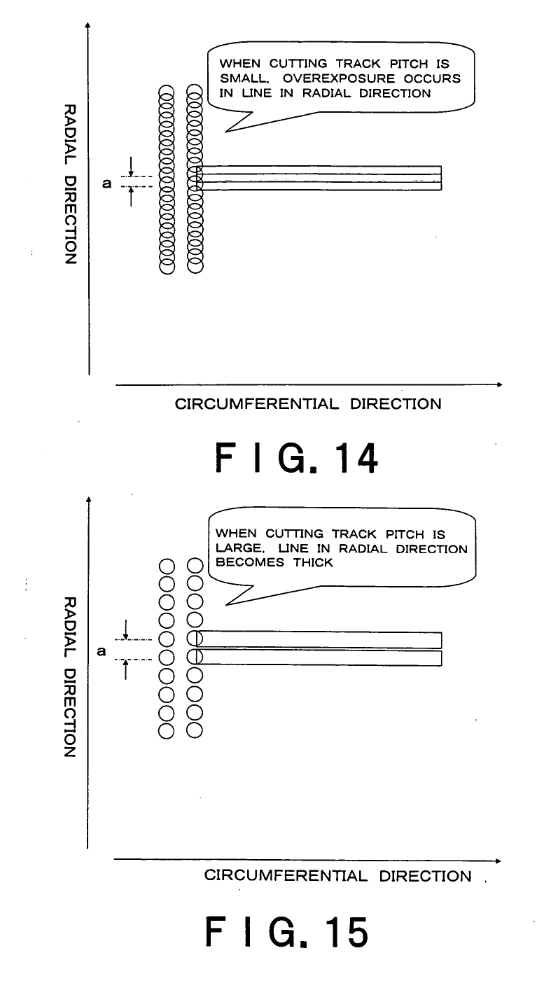

An electron beam irradiating method according to an embodiment of the present invention will be explained with reference to FIGS. 1 to 4F. An electron beam irradiating method according to the embodiment is used for a manufacturing method of a magnetic recording medium of a discrete type. As shown in FIG. 8, for example, the electron beam irradiating method is implemented using an electron beam irradiating apparatus of a stage continuous moving system provided with a moving mechanism for moving a stage in one horizontal direction and a rotating mechanism for rotating the stage. In the embodiment, it is desirable that the stage is rotated at a constant linear velocity in order to be kept an exposure amount even.

The electron beam irradiating method according to the embodiment is constituted so as to further perform exposure on a specific place which has been once exposed in the next exposing step and step(s) subsequent thereto, while changing a deflection intensity to an electron beam (a deflection amount). In the embodiment, the specific place is a portion corresponding to at least a groove of a discrete track, and it may include such another portion as, for example, a burst signal portion. The number of sectors is not limited to a specific one.

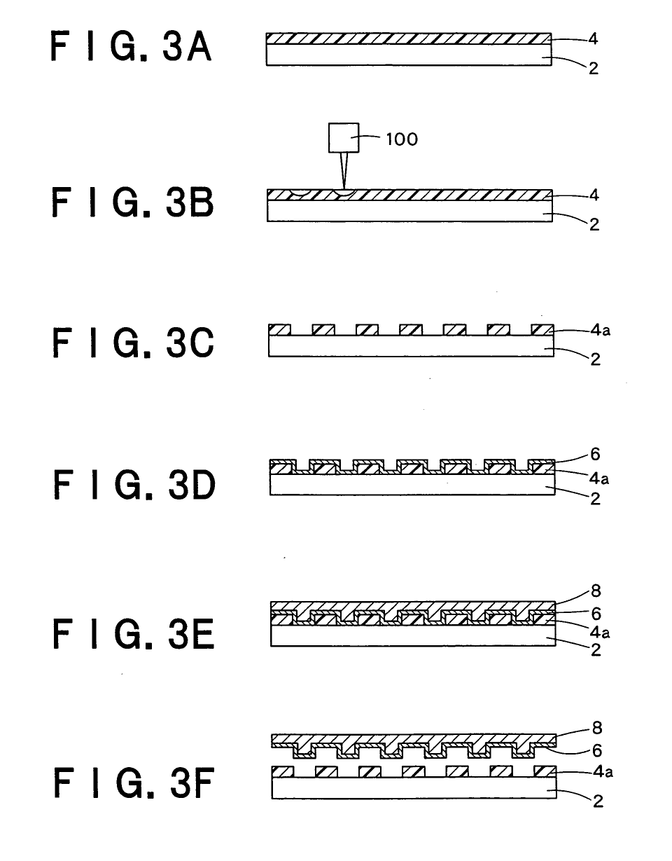

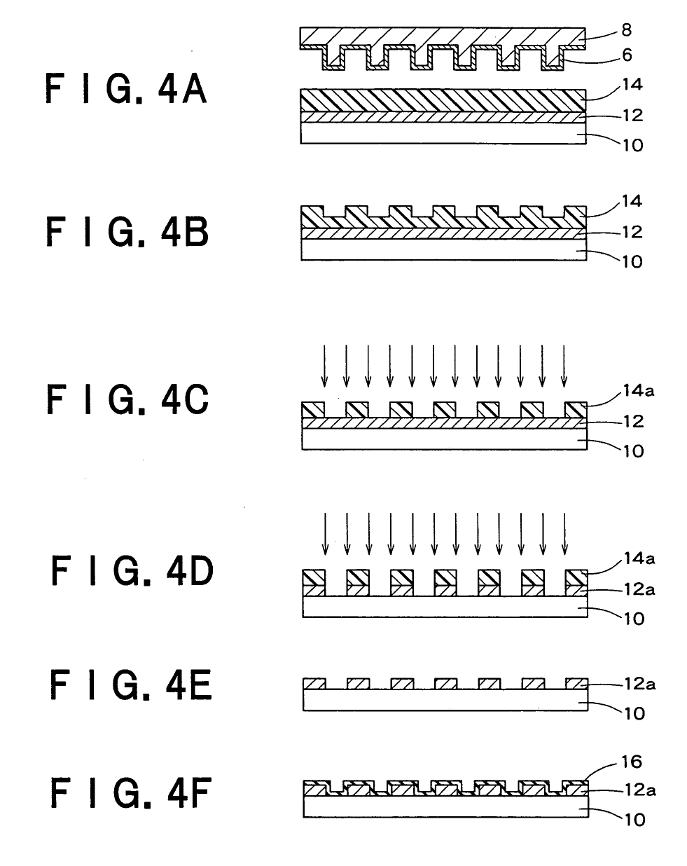

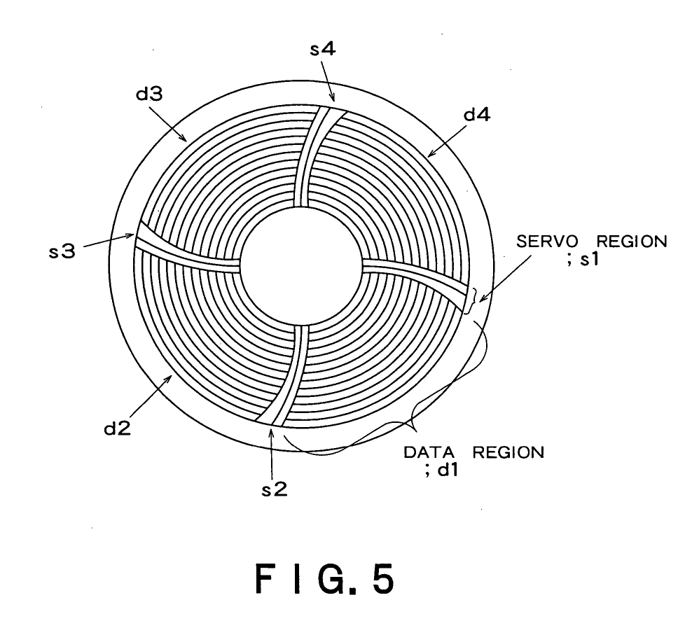

FIGS. 3A to 4F are diagrams showing manufacturing steps of a magnetic recording medium of a discrete type which is manufactured using the electron beam irradiating method according to the embodiment. The magnetic recording medium has such a constitution that four servo regions s1 to s4 and four data regions d1 to d4 are arranged, for example, as shown in FIG. 11.

Photosensitive resin (hereinafter, called “resist”) is applied on a substrate 2 (see FIG. 3A). The resist 4 may be of a positive type, of a negative type, of a chemical amplifying type, or a non-chemical amplifying type, but a positive type resist of a non-chemical amplifying type is desirable because of its excellent photosensitivity to an electron beam and its excellent resolution. Besides, material containing PMMA (polymethylmethacrylate) or novolac resin as a major component can be used. After the resist 4 is applied to the substrate, pre-braking is performed and the substrate is fed into a vacuum chamber in an electron beam irradiating apparatus, where the resist 4 is exposed by emission of an electron beam from an electron gun 100 (see FIG. 3B).

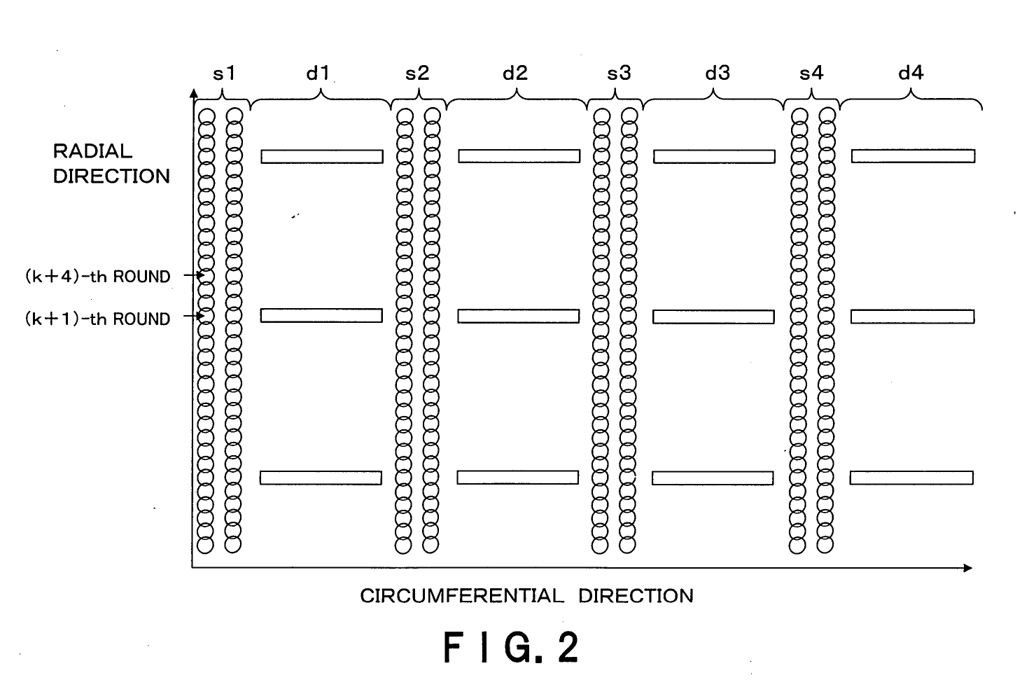

The electron beam irradiating method according to the embodiment of the invention is used in this exposing step. FIG. 1 is a diagram showing a relationship between the number of cutting rounds and a deflection amount of an electron beam according to the electron beam irradiating method of the embodiment, and FIG. 2 is a diagram showing an example of exposure performed at that time. As shown in FIG. 1, consideration is made on such a case that exposure for forming a groove of a track is performed in a certain round, for example, in the (k+1)-th round. Incidentally, a cutting track pitch (a feed amount for each round) is represented as “a”.

In the embodiment, as shown in FIG. 1, a deflection amount is increased in the (k+1)-th round according to advancing to a servo region s1, a data region d1, a servo region s2, a data region d2, a servo region s3, a data region d3, a servo region s4, and a data region d4. In the next round, namely, the (k+2)-th round, the deflection amount is returned back to zero at the servo region s1 and the deflection amount is increased according to advancing to the servo regions s2, s3, and s4, while the deflection amount is increased at the data regions d1, d2, d3, and d4 according to advancing to the data regions d1, d2, d3, and d4 so as to be positioned on an extension line of a deflection amount characteristic of the data regions d1, d2, d3, and d4 in the (k+1)-th round.

As shown in FIG. 1, the deflection amount is returned back to zero for a period from the (k+3)-th round and round(s) subsequent thereto to start of exposure for the next track groove again, where the deflection amount is increased according to advancing to the servo region s1, the data region d1, the servo region s2, the data region d2, the servo region s3, the data region d3, the servo region s4, and the data region d4, but the data regions d1, d2, d3, and d4 is blanked and electron beams are irradiated on only the servo regions s1, s2, s3, and s4.

In the embodiment, the deflection amount at a time of exposure termination to the data region d4 in the (k+1)-th round becomes equal to the cutting track pitch “a”, but the deflection amount at a time of exposure termination to the data region d4 in the (K+2)-th round becomes “2a”. The deflection amount at the servo regions s1, s2, s3, and s4 become the same in the (k+1)-th round and the (k+2)-th round.

Accordingly, the respective exposure positions at the data regions d1, d2, d3, and d4 exposed in the (k+1) round become equal to those at the data regions d1, d2, d3, and d4 in the (k+2) round, so that the same data regions d1, d2, d3, and d4 are exposed two times in a continuous manner. However, since the deflection amounts at the servo regions s1, s2, s3, and s4 in the (k+1)-th round are equal to those in the (k+2)-th round, the servo regions s1, s2, s3, and s4 exposed in the (k+1)-th round are different from those exposed in the (k+2)-th round, respectively.

Therefore, as shown in FIG. 2, exposure images at the data regions d1, d2, d3, and d4 constituting a groove on the data track portion in the (k+1)-th round and those in the (k+2)-th round overlap with each other, so that a thin groove can be formed. Incidentally, in order to expose the servo portion evenly in a radial direction to form a fine groove on the data track portion, it is preferable that drawing formation is made by six or more rounds per one data track and it is more preferable that drawing formation is made by 10 or more rounds per one data track.

Returning back to FIG. 3B again, the resist 4 is exposed in this manner. Thereafter, a resist pattern 4a is formed by developing the resist 4 using developer suitable for the resist 4, so that a resist original disk is produced (see FIG. 3C). Incidentally, a post-baking step may be included prior to the developing step.

Next, a thin conductive film 6 is formed on the resist pattern 4a of the resist original disk utilizing a Ni-sputtering or the like (see FIG. 3D). At that time, a film thickness of the resist pattern 4a is set such that a shape of a recess portion on the resist pattern 4a is sufficiently held by the conductive film 6. Thereafter, a Ni film 8 is embedded into the recess portion on the resist pattern 4a by plating so that the Ni film 8 is formed so as to have a desired film thickness (see FIG. 3E).

Next, the Ni film 8 is separated from the resist 4a and the substrate 2 so that a stamper 8 made from Ni is formed (see FIG. 3F). At that time, in order to remove the remaining resist on the stamper 8, such a processing as an oxygen RIE (reactive ion etching) is performed.

Next, as shown in FIG. 4A, a magnetic disk medium substrate is prepared by forming a magnetic layer 12 constituting a recording layer on the substrate 10 to apply resist 14 on the magnetic layer 12. Imprint is performed on the resist 14 applied on the magnetic disk medium substrate using the stamper 8 (see FIG. 4A), so that a pattern on the stamper 8 is transferred to the resist 14 (see FIG. 4B).

Next, the resist 14 is etched utilizing the pattern transferred on the resist 14 as a mask so that a resist pattern 14a is formed (see FIG. 4C). Subsequently, the magnetic layer 12 is applied with an ion milling process utilizing the resist pattern 14a as a mask (see FIG. 4D). The resist pattern 14 is then removed utilizing a dry etching process or chemicals so that a discrete magnetic layer 12a is formed (see FIG. 4E).

Next, a protective film 16 is formed on a whole surface (see FIG. 4F) so that a magnetic disk medium is completed.

A shape of a substrate formed on a pattern using electron beam irradiating method according to the embodiment is not limited to a specific one, but it may be preferably disk-like shape, for example, a silicon wafer. The disk-like substrate may include a notch or an orientation flat. Besides, a glass substrate, an Al-base alloy substrate, a ceramic substrate, a carbon substrate, a compound semiconductor substrate or the like may be used as the substrate. Amorphous glass or crystallization glass may be used for the glass substrate. The amorphous glass may be a soda lime glass, alumino-silicate glass, or the like. The crystallization glass may be lithium base crystallization glass or the like. As the ceramic substrate, a sintered body containing aluminum oxide, aluminum nitride, silicon nitride or the like as a major component, or material obtained by fiber-reinforcing each sintered body can be used. The compound semiconductor substrate may be GaAs, AlGaAs, or the like.

It is preferable in view of a system adopted for the magnetic disk medium that the shape of the magnetic disk medium is a disk shape, especially, a doughnut shape, but a size thereof is not limited to a specific one. However, it is desirable that the size is 3.5 inches or less such that a time of rendering performed using an electron beam does not increase excessively. It is further preferable that the size is 2.5 inches or less such that a pressing ability used at an imprinting time does not become excessive. It is more preferable in view of a mass productivity that the size is 1.8 inches or less, such as 0.85 inch or 1.8 inches, since the electron beam irradiating time is relatively short and a pressure required at an imprinting time is relatively low. The magnetic disk medium may have a single face or double faces to be used for recording.

The magnetic disk medium includes sectors obtained by partitioning an internal portion thereof to ring-like concentric tracks and sectioning these tracks for each constant angle, and the magnetic disk is attached to a spindle motor to be rotated so that recording/reproducing of various digital data elements is performed by a head. Therefore, user data tracks are arranged in a circumferential direction, while servo marks for position control are arranged in directions spanning the respective tracks. The servo mark includes regions such as a preamble portion, an address portion on which track or sector number information has been written, or a burst portion for relative position detection of a head to a track. In addition to these regions, a gap region(s) may be contained. In the embodiment, servo regions and data regions are arranged as shown in FIG. 11, but such a constitution may be employed that the servo regions s1, s2, s3, and s4 are formed in an arc shape extending along a locus of an arm, as shown in FIG. 5.

In view of improvement in recording density, it is required that a track pitch is made narrower. Since it is necessary to form a non-magnetic portion serving as a separation portion for a user data region portion and a magnetic portion serving as a recording region for data, form an address bit for a corresponding servo region, or form a burst mark or the like even in one track, it is required to perform rendering so as to form one track in several rounds to several tens rounds during cutting process. Here, when the number of cutting rounds is small, a shape resolution becomes low so that a pattern shape can not be reflected excellently. On the other hand, when the number of cutting rounds is large, such a problem arises that control signals are complicated and storage capacity must be made large. Therefore, it is desirable that one track is formed at the number of cutting rounds which is at least 6 to at most 36. It is advantageous in view of pattern arrangement design that the number of cutting rounds is a number with many aliquots.

Since the sensitivity of the resist to be exposed is even within a plane, it is desirable that a stage in the electron beam irradiating apparatus is rotated while a linear velocity thereof is kept constant. A deflection amount for overwriting may be a width allowing a proper deflection or less. For example, when one user data region is defined by a pitch of 300 nm, a cutting track pitch becomes 300÷12=25 nm in order to form one track by performing cutting 12 rounds. Therefore, when overlapping is performed one time in rendering, the deflection amount may be at most 25 nm, and when overlapping is performed two times, the deflection amount may be at most 50 nm to ±25 nm (deflection directions are revered to each other in the two overlapping times). When a complete overlapping is not required, the maximum deflection may be less than the above numerical value.

When photosensitive resin to be used is of the so-called positive type where an exposed portion is developed and removed, it is required to perform overwriting (n+1) or more times under such a condition that, when development is performed after exposure is performed n times at a linear velocity V (m/s), a film thickness removed by the exposure and development does not reach a film thickness t to be removed by the exposure and development and when development is performed after exposure is performed (n+1) times at a linear velocity V (m/s), a film thickness removed by the exposure and development reaches the film thickness t to be removed.

On the contrary, when photosensitive resin to be used is of the so-called negative type where a non-exposed portion is developed and removed, it is required to perform overwriting (n+1) or more times under such a condition that, when development is performed after exposure is performed n times at a linear velocity V (m/s), the remaining film on the exposed portion to be left is not formed to have a film thickness t and when development is performed (n+1) times at a linear velocity V (m/s), the remaining film to be left can be formed to have the film thickness t.

For example, in a processing where an electron beam with a beam diameter of 50 nm and a current value of 15 nA is used, a film of a positive type resist (for example, ZEP-520 (produced by NIPPON ZEON CORP.)) formed on a silicon substrate to have a film thickness of 100 nm is exposed at a linear velocity of 0.7 m/s and is developed by dipping the silicon substrate in developer (for example, ZED-N50 (produced by NIPPON ZEON CORP.)) for 90 seconds, the developed film is then rinsed by dipping the silicon substrate in rinsing liquid (for example, ZMD-B (NIPPON ZEON CORP.)) for 90 seconds, and the silicon substrate is dried by air blowing, when exposure is performed only one time, the remaining film is left on the exposed portion, but it is not left when exposure is performed two or more times. Therefore, overwriting must be performed two or more times. An upper limit of the number of times of overwriting is not limited to a specific one, but it is undesirable that the number of times of overwriting exceeds double of the number of times required in view of such a purpose that a groove with thinner line width is formed by performing overlapping drawing.

As described above, according to the embodiment, as shown in FIG. 2, a groove on a data region can be formed to be thinner, as compared with the conventional technique. Therefore, it is made possible to increase a user data region and it is also made possible to dense a track pitch, so that a recording density can be improved. Further, a taper of a groove portion on a magnetic recording medium takes a rising shape, so that a signal intensity is increased and an S/N ratio is reduced, and pressing at imprinting time is made easy.

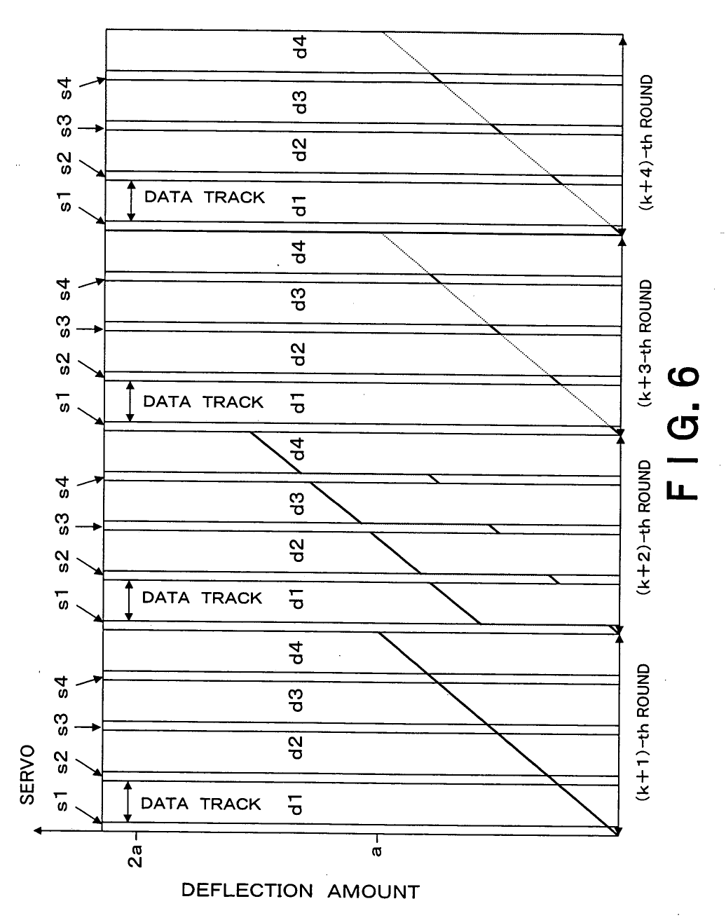

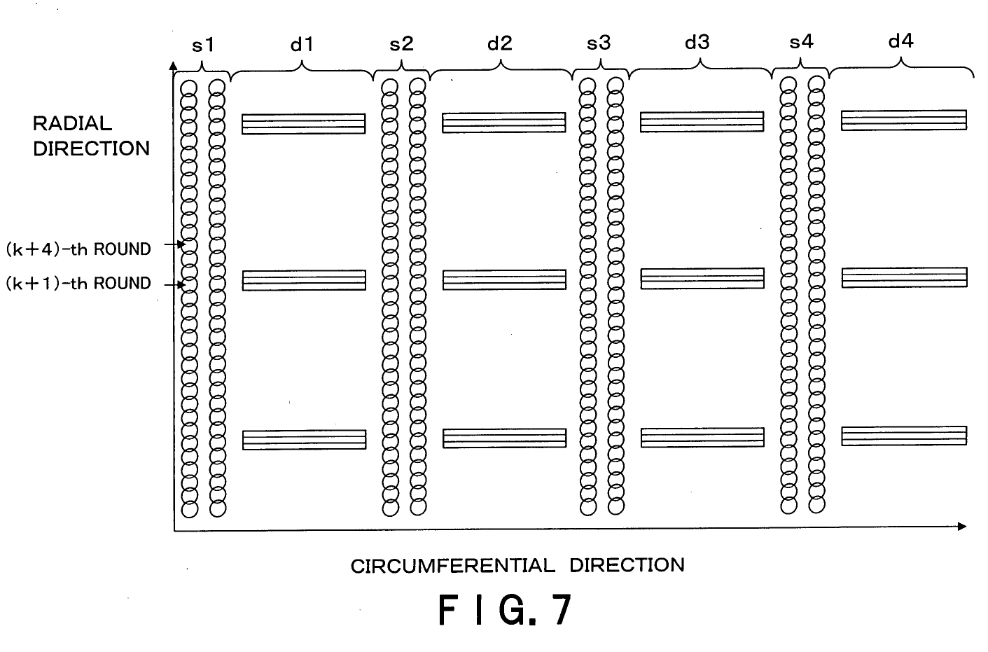

In the embodiment, it is not required necessarily to cause the (k+2)-th track region to overlap with the (k+1)-th track region completely, but such a constitution may be employed, as shown in FIG. 7, that the both are caused to overlap with each other to a certain extent by performing such deflection as shown in FIG. 6. Such a constitution is advantageous, since a groove width to be formed can be changed by adjusting a magnitude of the deflection. In FIG. 6, a deflection amount of an electron beam at a starting time of exposure to the (k+2)-th data track region d1 is set to be larger than zero and be smaller than the cutting track pitch “a”. When exposure images are caused to partially overlap with each other in this manner, it is desirable for pattern formation to adjust deflection of an electron beam such that an exposure amount of a pattern becomes symmetrical regarding a section in a radial direction.

Regarding a stage, an optical system for scanning an electron beam, and signals for activating them in the electron beam irradiating apparatus, it is at least required that a point where a deflection intensity is changed and a signal for the deflection intensity, a signal about a blanking point and a blanking signal, a stage activating signal for movement control in a radial direction and in a rotational direction are synchronized with one another.

Next, Examples of the present invention will be explained.

### EXAMPLE 1

A manufacturing method of a magnetic recording medium according to an example 1 of the present invention will be explained with reference to FIGS. 3A and 4F.

An electron beam irradiating apparatus with acceleration voltage of 50 kV having an electron gun, a condenser lens, an objective lens, a blanking electrode, and an electron gun emitter of a ZO/W TFE (thermal field emission) type provided with a deflector which performs 20 nm deflection when applied with a voltage of 20 mV and performs 40 nm deflection when applied with a voltage of 40 mV was used.

On the other hand, after resist ZEP-520 produced by NIPPON ZEON CORP. was diluted to two times and the diluted resist was filtrated by a membrane filter with 0.2 μm mesh size, the filtrated resist was spin-coated on a 8-inch silicon wafer substrate 2 HMDS-processed, and the wafer substrate is pre-baked at a temperature of 200° C. for three minutes, so that a resist 4 with a film thickness of 0.1 μm was formed (see FIG. 3A).

The substrate 2 was transported to a predetermined position in the electron beam irradiating apparatus by a transporting system thereof, and it was exposed under vacuum for obtaining a concentric type pattern satisfying the following conditions (see FIG. 3B).

Exposed portion radius: 4.8 mm to 10.2 mm

Number of Sectors: 120

Track pitch: 300 nm

Feeding amount: 20 nm

Address portion bit: 0 to 1000

Track portion bit: 1001 to 9999

Since the track pitch is 15 times the feeding amount, one track is formed by performing exposure 15 rounds. A concentric circle was drawn while increasing deflection intensity from 0 mV to 20 mV during one rotation in an ordinary round. Regarding (15k+8) (k denoted 0 or a natural numeral) rounds, however, exposure was conducted in m (m denoted 1 to 120) sectors while increasing the deflection intensity in an address portion from 20×(m−1)/120 [mV] to 20×(m−1)/120+20×1/120×1000/10000 [mV] and increasing the deflection intensity in a track portion from 20+20×(m−1)/120+20×1/120×1000/10000 [mV] to 20+20×m/120 [mV].

Incidentally, the address portion included a preamble pattern, a burst pattern, a sector and track address pattern, and a gap pattern.

A signal source was used which could generate a signal for forming a pattern and a signal to be fed to the stage driving system which were synchronized with deflection control of an electron beam. The stage was rotated at CLV (constant linear velocity) of a linear velocity of 500 mm/s and it was also moved in a rotational radial direction during exposure.

After exposure, the silicon wafer substrate 2 was developed by dipping the same in developer (for example, ZED-N50 (produced by NIPPON ZEON CORP.)), the developed silicon wafer substrate was then rinsed by dipping the same in rinsing liquid (for example, ZMD-B (produced by NIPPON ZEON CORP.)) for 90 seconds, and the rinsed wafer substrate was dried by air blowing, so that a resist original disk with a rugged surface was produced (see FIG. 3C).

An electrical conductive film 6 was formed on the resist original disk by a sputtering process. Pure nickel was used as target, and sputtering was conducted for 40 seconds under application of DC power of 400 W within a chamber which was vacuumed to 8×10⁻³ Pa and then introduced with argon to be adjusted to 1 Pa, so that a conductive film with a thickness of 30 nm was obtained (see 3D).

The resist original disk with the conductive film 6 was plated for 90 minutes using nickel sulfamate plating liquid (NS-160 produced by SHOWA KAGAKU CO., LTD) (see FIG. 3E).

Plating bath conditions were as follows:

Nickel sulffamate nickel: 600 g/L

Boric acid: 40 g/L

Interfacial active agent (sodium lauryl sulfate): 0.15 g/L

Liquid temperature: 55° C.

pH: 4.0

Current density: 20 A/dm²

A thickness of the plated film 8 was 300 μm. Thereafter, a stamper 8 provided with the conductive film 6, the plated film 8 and the resist residue was obtained by peeling off the plated film 8 from the resist original disk (see FIG. 3F).

The resist residue was removed by an oxygen plasma ashing process. The oxygen plasma ashing was conducted for 20 minutes with a power of 100 W within a chamber which was introduced with oxygen gas at a flow rate of 100 ml/min to be adjusted to a vacuum of 4 Pa. A father stamper 8 provided the conductive film and the plated film was obtained. Thereafter, an imprint stamper 8 was obtained by removing an unnecessary portion(s) of the obtained stamper 8 through a punching process.

After the stamper 8 was subjected to ultrasonic cleaning for 15 minutes using acetone, it was dipped for 30 minutes in solution obtained by diluting fluoroalkylsilane (CF₃(CF₂)₇CH₂CH₂Si(OMe)₃) (TSL 8233 produced by GE TOSHIBA SILICONE CORP.) to 5% solution using ethanol. After the solution on the stamper 8 was blown off by a blower, it was annealed at a temperature of 120° C.

On the other hand, as a substrate to be worked or processed, a magnetic recording layer 12 was formed on a doughnut type glass substrate 10 with a diameter of 0.85 inch by sputtering process, and novolac-base resist (S1801 produced by ROHM & HAAS CORP.) was spin-coated on the recording layer 12 at a rotational velocity of 3800 rpm (see FIG. 4A). Thereafter, the pattern on the stamper 8 was transferred on the resist 14 by pressing the stamper 8 on the substrate for 1 minute with a weight of 2000 bar (see FIG. 4B). After UV irradiation was conducted to the resist 14 transferred with the pattern for 5 minutes, the resist 14 was heated at 160° C. for 30 minutes.

An oxygen RIE was performed to the substrate 10 imprinted in the above manner under an etching pressure of 2 mTorr using an ICP (inductively coupled plasma) etching apparatus (see FIG. 4C), and the recording layer 12 was then etched by an Ar ion milling (see FIG. 4D). After etching to the magnetic layer 12, oxygen RIE was conducted with a power of 400 W and under a pressure of 1 Torr in order to remove the etching mask 14a made from resist. After removal of the etching mask 14a, a DLC (diamond like carbon) film with a thickness of 3 nm was formed as the protective film 16 by a CVD (chemical vapor deposition) process. Further, lubricant was applied to the protective film to have a thickness of 1 nm.

A width of a groove on a track portion on the medium imprinted and processed in this manner was 80 nm.

### EXAMPLE 2

A manufacturing method of a magnetic recording medium according to an example 2 of the present invention will be explained. In the example 2, a magnetic recording medium was manufactured like the example 1 except for deflection intensity of an electron beam. Regarding the deflection intensity of an electron beam in this example, exposure was conducted in m (m denoted 1 to 120) sectors about (15k+8) (k denoted 0 or a natural number) round while increasing the deflection intensity in an address portion from 20×(m−1)/120 [mV] to 20×(m−1)/120+20×1/120×1000/10000 [mV] and increasing the deflection intensity in a track portion from 15+20×(m−1)/120+20×1/120×1000/10000 [mV] to 15+20×m/120 [mV].

A width of a groove of the track portion of a medium imprinted and processed was 100 nm, which was wider than that in the example 1.

### EXAMPLE 3

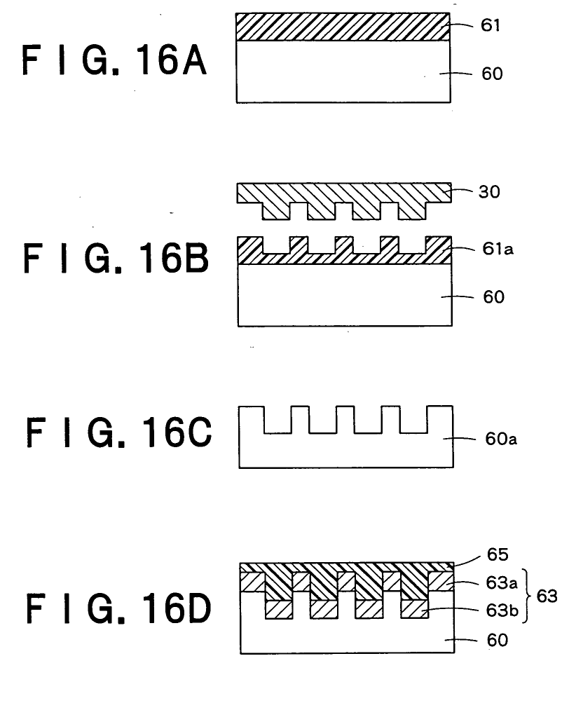

Next, a manufacturing method of a magnetic recording medium according to an example 3 of the invention will be explained with reference to FIGS. 16A to 16D. A magnetic recording medium manufactured by the manufacturing method of the example 3 was a magnetic recording medium (a substrate-patterned discrete media) of a substrate processing type.

An imprint stamper 30 was manufactured using a process similar to the process shown in FIGS. 3A to 3F, especially, utilizing the irradiating method of the present invention in the step shown in FIG. 3B.

Next, a rugged substrate was manufactured using an imprint lithography process in the following manner. As shown in FIG. 16A, resist 61 for imprinting was applied on the substrate 60. Subsequently, as shown in FIG. 16B, the stamper 30 was opposed to the resist 61 on the substrate 60 a projection pattern on a surface of the stamper 30 was transferred on a surface of the resist 61 by pressing the stamper 30 onto the resist 61 by application of a pressure. Thereafter, the stamper 30 was detached. Thereby, a resist pattern 61a where ruggedness was formed on the resist 61 was obtained (see FIG. 16B).

Next, a substrate 60a formed with the rugged pattern was obtained by etching the substrate 60 using the resist pattern 61a as a mask. Thereafter, the resist pattern 61a was removed (see FIG. 16C).

Subsequently, as shown in FIG. 16D, a magnetic film 63 suitable for vertical recording was formed on the substrate 60a. At that time, a magnetic film formed on a projecting portion of the substrate 60a served as a projection magnetic substance portion 63a and a magnetic film formed in a recessed portion of the substrate 60a served as a recess magnetic substance portion 63b. It is preferable that the magnetic film 63 is formed as a stacked film of a soft magnetic foundation layer and a ferromagnetic recording layer. Further, a magnetic recording medium was manufactured by providing a protective film 65 made from carbon on the magnetic film 63 and further applying lubricant on the protective film 65.

A magnetic substance portion and a non-magnetic substance portion of the magnetic recording medium (magnetic film-patterned discrete track media) of the magnetic substance processing type described with reference to FIG. 4F correspond to the projection magnetic substance portion 63a and the recess magnetic substance portion 63b in the example, respectively. Functions of both the portions 63a and 63b are the same in the magnetic recording medium.

### COMPARATIVE EXAMPLE

A magnetic recording medium was manufactured in the same manner as the example 1 except for deflection intensity. In the comparative example, a concentric circle was drawn in all rounds while gradually increasing deflection intensity from 0 mV to 20 mV during one rotation. A width of a groove on a track portion of a medium imprinted and processed was 150 nm.

When a pressure at an imprinting time was 2000 bar like the first embodiment, pressing became insufficient, which resulted in occurrence of pressing unevenness. When a pressure of 2200 bar was applied at the imprinting time, excellent imprint could be obtained like the first and second embodiments.

According to the respective embodiment of the present invention, since a fine pattern can be formed, it is made possible to improve a recording density and increase signal intensity using each embodiment for manufacturing a magnetic medium.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concepts as defined by the appended claims and their equivalents.

What is claimed is:

1. An electron beam irradiating method which irradiates an electron beam on a resist film to perform irradiating using an electron beam irradiating apparatus provided with a moving mechanism which moves a state on which a substrate applied with the resist film is put in one horizontal direction, and a rotating mechanism which rotates the stage, the method comprising:
exposing a portion once exposed while changing a deflection amount of the electron beam at least one time in the next round and rounds subsequent thereto, when exposure is performed while a deflection amount of an electron beam is being gradually changed so as to draw a concentric circle for each round.

2. An electron beam irradiating method according to claim 1, wherein the deflection amount of an electron beam when exposure is performed while changing a deflection amount of the electron beam at least one time in the next round and rounds subsequent thereto is changed such that an exposed image before the portion once exposed is again exposed substantially overlaps with an exposed image obtained by performing exposure while changing the deflection amount of electron beam at least one time in the next round and one round subsequent thereto.

3. An electron beam irradiating method according to claim 1, wherein the deflection amount of an electron beam when exposure is performed while changing the deflection amount of the electron beam at least one time in the next round and rounds subsequent thereto is changed such that an exposed image before the portion once exposed is again exposed partially overlaps with an exposed image obtained by performing exposure while changing the deflection amount of electron beam at least one time in the next round and rounds subsequent thereto.

4. An electron beam irradiating method according to claim 3, wherein, when one pattern is exposed in a circumferential direction by performing exposure in at least three rounds while changing the deflection amount of the electron beam, exposure is performed while changing the deflection amount of the electron beam such that an exposure amount of the pattern becomes symmetrical regarding a section in a radial direction.

5. An electron beam irradiating method according to claim 1, wherein the stage is rotated at a constant linear velocity.

6. A manufacturing method of a magnetic recording medium that manufactures a magnetic recording medium having at least a servo region and a data region, where adjacent tracks for the data region are separated from each other by a non-magnetic portion, the manufacturing method being implemented according to an imprint process and comprising:
conducting a portion of a resist original disk for manufacturing a stamper used for the imprint process which corresponds to the data region portion by utilizing an electron beam irradiating method according to claim 1.

7. A manufacturing method of a magnetic recording medium according to claim 6, wherein the magnetic recording medium is formed in a drawing manner in at least six rounds including a round where drawing is not performed per one data track.

8. A manufacturing method of a magnetic recording medium according to claim 6, wherein the deflection amount of an electron beam when exposure is performed while changing a deflection amount of the electron beam at least one time in the next round and rounds subsequent thereto is changed such that an exposed image before the portion once exposed is again exposed substantially overlaps with an exposed image obtained by performing exposure while changing the deflection amount of electron beam at least one time in the next round and one round subsequent thereto.

9. A manufacturing method of a magnetic recording medium according to claim 6, wherein the deflection amount of an electron beam when exposure is performed while changing the deflection amount of the electron beam at least one time in the next round and rounds subsequent thereto is changed such that an exposed image before the portion once exposed is again exposed partially overlaps with an exposed image obtained by performing exposure while changing the deflection amount of electron beam at least one time in the next round and rounds subsequent thereto.

10. A manufacturing method of a magnetic recording medium according to claim 9, wherein, when one pattern is exposed in a circumferential direction by performing exposure in at least three rounds while changing the deflection amount of the electron beam, exposure is performed while changing the deflection amount of the electron beam such that an exposure amount of the pattern becomes symmetrical regarding a section in a radial direction.

11. A manufacturing method of a magnetic recording medium according to claim 6, wherein the stage is rotated at a constant linear velocity.

12. A manufacturing method of a magnetic recording medium according to claim 6, further comprising:
forming a resist layer on a substrate formed with a magnetic layer;
forming a resist pattern transferred with a rugged pattern of the stamper on the resist layer by performing imprinting using the stamper; and
patterning the magnetic layer using the resist pattern as a mask.

13. A manufacturing method of a magnetic recording medium that manufactures a magnetic recording medium having at least a servo region and a data region, where adjacent tracks for each of the data region are separated from each other by ruggedness of a magnetic substance, the manufacturing method comprising:
conducting a portion of a resist original disk for manufacturing a stamper used for the imprint process which corresponds to the data region portion by utilizing an electron beam irradiating method according to claim 1.

14. A manufacturing method of a magnetic recording medium according to claim 13, wherein the magnetic recording medium is formed in a drawing manner by at least six rounds including a round where drawing is not performed per one data track.

15. A manufacturing method of a magnetic recording medium according to claim 13, wherein the deflection amount of an electron beam when exposure is performed while changing a deflection amount of the electron beam at least one time in the next round and rounds subsequent thereto is changed such that an exposed image before the portion once exposed is again exposed substantially overlaps with an exposed image obtained by performing exposure while changing the deflection amount of electron beam at least one time in the next round and one round subsequent thereto.

16. A manufacturing method of a magnetic recording medium according to claim 13, wherein the deflection amount of an electron beam when exposure is performed while changing the deflection amount of the electron beam at least one time in the next round and rounds subsequent thereto is changed such that an exposed image before the portion once exposed is again exposed partially overlaps with an exposed image obtained by performing exposure while changing the deflection amount of electron beam at least one time in the next round and rounds subsequent thereto.

17. A manufacturing method of a magnetic recording medium according to claim 16, wherein, when one pattern is exposed in a circumferential direction by performing exposure in at least three rounds while changing the deflection amount of the electron beam, exposure is performed while changing the deflection amount of the electron beam such that an exposure amount of the pattern becomes symmetrical regarding a section in a radial direction.

18. A manufacturing method of a magnetic recording medium according to claim 13, wherein the stage is rotated at a constant linear velocity.

19. A manufacturing method of a magnetic recording medium according to claim 13, further comprising:
forming a resist layer on a substrate formed with a magnetic layer;
forming a resist pattern transferred with a rugged pattern of the stamper on the resist layer by performing imprinting using the stamper; and
patterning the magnetic layer using the resist pattern as a mask.

20. A manufacturing method of a magnetic recording medium according to claim 13, further comprising:
forming a resist layer on a substrate;
forming a resist pattern transferred with a rugged pattern of the stamper on the resist layer by performing imprinting using the stamper;
patterning the substrate using the resist pattern as a mask; and
forming a magnetic film on a rugged portion on the substrate after removing the resist pattern.
